(19) United States
(12) Patent Application Publication
(10) Pub. No.: US 2025/0331799 A1
(43) Pub. Date: Oct. 30, 2025

(54) RADIATION IMAGE PROCESSING APPARATUS, RADIATION IMAGE PROCESSING SYSTEM, RADIATION IMAGE PROCESSING METHOD, AND STORAGE MEDIUM

(71) Applicant: Konica Minolta, Inc., Tokyo (JP)

(72) Inventor: Ryohei ITO, Tokyo (JP)

(21) Appl. No.: 19/176,301

(22) Filed: Apr. 11, 2025

(30) Foreign Application Priority Data
Apr. 25, 2024 (JP) 2024-071809

(51) Int. Cl. A61B 6/00; A61B 6/46

(52) U.S. Cl. CPC A61B 6/5235; A61B 6/463; A61B 6/5258

(57) ABSTRACT

A radiation image processing apparatus includes a hardware processor that obtains radiation images of a subject captured under imaging conditions different from one another, and an image processor that corrects a pixel value of at least one radiation image of the radiation images, and combines the radiation images to obtain one composite image. Each of the radiation images has an image overlap area with one or more other radiation images of the radiation images. The image processor corrects the pixel value of the at least one radiation image such that a maximum value of the pixel value of the at least one radiation image is equal to or smaller than a maximum value of an image gradation.

### CROSS-REFERENCE TO RELATED APPLICATIONS

The entire disclosure of Japanese Patent Application No. 2024-071809, filed on Apr. 25, 2024, is incorporated herein by reference in its entirety.

### BACKGROUND OF THE INVENTION

### Technical Field

The present invention relates to a radiation image processing apparatus, a radiation image processing system, a radiation image processing method, and a storage medium.

### Description of Related Art

There is known a method of generating a composite image by combining images obtained by changing an emission direction of a radiation source in a body axis direction (so-called long length imaging by swinging).

In the method, it is necessary to expose a subject to radiation multiple times. Therefore, adverse effects on the health of the subject may occur. Therefore, for example, in Japanese Patent No. 5759405 and Japanese Patent No. 4754812, there is disclosed a method of suppressing the influence of exposure by changing an imaging condition each time an image is captured, and automatically correcting the pixel value of each image at the time of image combination.

### SUMMARY OF THE INVENTION

However, in Japanese Patent No. 5759405 and Japanese Patent No. 4754812, there is not disclosed any specific method for correcting the pixel value. Therefore, the pixel value of the composite image may become equal to or more than the maximum value of gradation (so-called density saturation) and cause blocked-up shadows, which makes it difficult to interpret the image.

The present invention has been made in view of these circumstances. An object of the present invention is to provide a radiation image processing apparatus, a radiation image processing system, a radiation image processing method, and a storage medium storing a program that are capable of obtaining a composite image that can be easily interpreted.

To achieve at least one of the abovementioned objects, according to an aspect of the present invention, a radiation image processing apparatus reflecting one aspect of the present invention includes:
a hardware processor that obtains radiation images of a subject captured under imaging conditions different from one another; and
an image processor that corrects a pixel value of at least one radiation image of the radiation images, and combines the radiation images to obtain one composite image,
wherein each of the radiation images has an image overlap area with one or more other radiation images of the radiation images, and
wherein the image processor corrects the pixel value of the at least one radiation image such that a maximum value of the pixel value of the at least one radiation image is equal to or smaller than a maximum value of an image gradation.

According to an aspect of the present invention, a radiation image processing system reflecting one aspect of the present invention includes:
the above-described radiation image processing apparatus;
a radiation emission apparatus that emits radiation; and
a radiographic imaging apparatus that generates a radiation image by receiving the radiation emitted by the radiation emission apparatus.

According to an aspect of the present invention, a radiation image processing method reflecting one aspect of the present invention includes:
obtaining radiation images of a subject captured under imaging conditions different from one another;
correcting a pixel value of at least one radiation image of the radiation images, and
combining the radiation images to obtain one composite image,
wherein each of the radiation images has an image overlap area with one or more other radiation images of the radiation images, and
wherein the correcting includes correcting the pixel value of the at least one radiation image such that a maximum value of the pixel value of the at least one radiation image is equal to or smaller than a maximum value of an image gradation.

According to an aspect of the present invention, a storage medium reflecting one aspect of the present invention stores a program causing a computer to:
obtain radiation images of a subject captured under imaging conditions different from one another;
correct a pixel value of at least one radiation image of the radiation images, and
combine the radiation images to obtain one composite image,
wherein each of the radiation images has an image overlap area with one or more other radiation images of the radiation images, and
wherein the program causes the computer to correct the pixel value of the at least one radiation image such that a maximum value of the pixel value of the at least one radiation image is equal to or smaller than a maximum value of an image gradation.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a schematic diagram of a radiation image processing system;

FIG. 2 is a block diagram of a console;

FIG. 3 is a flowchart of a composite image obtaining process;

FIG. 4 is a diagram illustrating an example of a plurality of radiation images;

FIG. 5 is a diagram illustrating an example of one composite image;

FIG. 6 is a diagram illustrating an example of a display part on which a composite image is displayed; and

FIG. 7 is a diagram illustrating an example of a composite image according to a modification example.

### DETAILED DESCRIPTION

Hereinafter, one or more embodiments of the present invention will be described with reference to the drawings. However, the scope of the present invention is not limited to the following embodiments or the drawings.

### Radiation Image Processing System

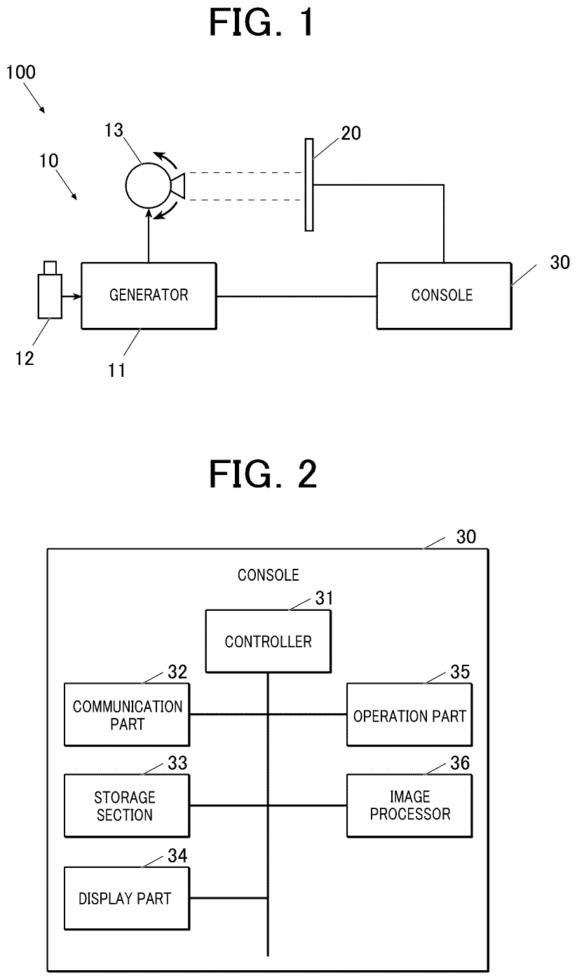

First, a radiation image processing system 100 according to the present embodiment will be described. FIG. 1 is a schematic diagram of a radiation image processing system 100.

As illustrated in FIG. 1, the radiation image processing system 100 includes a radiation emission apparatus 10, a radiographic imaging apparatus 20, and a console 30 that is a radiation image processing apparatus. The apparatuses constituting the radiation image processing system 100 are communicably connected to each other via, for example, a communication network (local area network (LAN), wide area network (WAN), the Internet, or the like).

Note that the radiation image processing system 100 may be communicably connected to a not-illustrated system. The system is, for example, a hospital information system (HIS), a radiology information system (RIS), a picture archiving and communication system (PACS), or the like.

### Radiation Emission Apparatus

The radiation emission apparatus 10 emits radiation in a form corresponding to the type of radiation image. The radiation emission apparatus 10 includes a generator 11, an exposure switch 12, and a radiation source 13.

### Generator and Exposure Switch

The generator 11 applies a voltage corresponding to a preset imaging condition to the radiation source 13 in response to an operation on the exposure switch 12.

### Radiation Source

The radiation source 13 includes a tube and a filament (not illustrated). The radiation source 13 is arranged at a position facing the radiographic imaging apparatus 20 with a subject interposed therebetween. When a voltage is applied from the generator 11, the filament emits an electron beam corresponding to the voltage to the rotary anode. The rotary anode irradiates the subject with a dose of radiation (X-rays) corresponding to the intensity of the electron beam. The radiation source 13 can change the direction of an emission port of radiation by rotating around a rotation axis parallel to an X-axis direction, a Y-axis direction orthogonal to the X-axis, and a Z-axis direction orthogonal to the X-axis and the Y-axis.

Note that although the generator 11, the exposure switch 12, and the radiation source 13 are illustrated as separate and independent components in FIG. 1, they are not limited to this. The generator 11, the exposure switch 12, and the radiation source 13 may be integrated. Furthermore, the exposure switch 12 may be connected to a operation console (not illustrated) instead of the generator 11. The radiation emission apparatus 10 may be installed in an imaging room or may be of a portable type that is configured to be movable by being incorporated in, for example, a medical cart.

### Radiographic Imaging Apparatus

The radiographic imaging apparatus 20 detects radiation emitted from the radiation source 13 and transmitted through the subject to capture a radiation image. The radiographic imaging apparatus 20 includes a flat panel detector (FPD). The radiographic imaging apparatus 20 includes, for example, a glass substrate. The radiographic imaging apparatus 20 detects, in accordance with its intensity, radiation (X-rays) that has been emitted from the radiation emission apparatus 10 to a predetermined position on a substrate and has passed through at least the subject. In the radiographic imaging apparatus 20, detection elements (pixels) that convert detected radiation into electric signals and accumulate the electric signals are arranged in a matrix. Each pixel includes a switching section such as a thin film transistor (TFT), for example. The radiographic imaging apparatus 20 controls the switching section of each pixel on the basis of an image reading condition input from the console 30, switches reading of the electric signal accumulated in each pixel, and reads the electric signal accumulated in each pixel. By the control, the radiographic imaging apparatus 20 obtains image data (frame image). Then, the radiographic imaging apparatus 20 outputs the obtained image data to the console 30.

### Console

The console 30 receives input of the imaging condition to be set in at least one of the radiation emission apparatus 10 and the radiographic imaging apparatus 20. The console 30 is a dedicated apparatus such as a PC. The method for inputting the imaging condition to the console 30 is not particularly limited. Examples of the method include a user operation, obtaining it from another system (HIS, RIS, or the like), and an another user (e.g., technician) operation. The configuration of the console 30 will be detailed later.

In the radiation image processing system 100 configured in such a manner, when a user operates an emission instruction switch 111, the radiation source 13 irradiates the subject with radiation under the imaging condition in accordance with imaging order information described later. Then, the radiographic imaging apparatus 20 located behind the subject receives the radiation transmitted through the subject, reads image data, and transmits the image data to the console 30.

### Detailed Configuration of Console

The detailed configuration of the console 30 will be described. FIG. 2 is a block diagram of the console 30. The console 30 includes a controller 31 (hardware processor), a communication part 32, a storage section 33, a display part 34 (display), an operation part 35, and an image processor 36. These components of the console 30 are electrically connected to each other via a bus or the like. The console 30 functions as a radiation image processing apparatus that receives various kinds of input related to a composite image obtaining process, which will be described later, and performs various kinds of processing.

### Controller

The controller 31 includes a central processing unit (CPU), a random access memory (RAM), and the like. The CPU reads various programs stored in the storage section 33 and loads the programs to the RAM. The CPU performs various processes in accordance with the loaded programs. With the above configuration, the controller 31 centrally controls the operation of each component of the console 30.

### Communication Part

The communication part 32 is constituted by a communication module or the like. The communication part 32 transmits and receives various signals and various data to and from other apparatuses such as the radiation emission apparatus 10 and the radiographic imaging apparatus 20 connected via the communication network.

### Storage Section

The storage section 33 is constituted by a nonvolatile semiconductor memory, a hard disk, and/or the like. The storage section 33 stores various programs to be executed by the controller 31, parameters required for executing the programs, and so forth. The storage section 33 may be capable of storing image data of radiation images. Furthermore, the storage section 33 stores imaging order information transmitted from the RIS or the like.

The imaging order information includes information on the subject who is a patient, examination information, and the imaging condition. The examination information includes an examination ID, an imaging site, an examination date, and the like. The imaging condition includes various conditions related to the amount of radiation emitted by the radiation emission apparatus 10. Examples thereof include the angle of the radiation source 13 (i.e., imaging site), positioning (PA, LAT, etc), tube voltage (kV), tube current (mA), emission/irradiation time (ms), current-time product (mAs value), and focus-to-radiographic imaging apparatus distance (SID).

### Display Part

The display part 34 displays various screens. The display part 34 is configured by, for example, a liquid crystal display (LCD), an electronic luminescent display (ELD), a cathode ray tube (CRT), or the like. The display part 34 displays lists, radiation images, and the like in accordance with image signals received from the controller 31.

### Operation Part

The operation part 35 is configured to be operable by the user. The operation part 35 is a keyboard including cursor keys, number input keys, and various function keys, a pointing device (such as a mouse), a touch screen superimposed on the surface of the display part 34, or the like. The operation part 35 outputs control signals corresponding to operations made by the user to the controller 31.

### Image Processor

The image processor 36 performs image processing on radiation images obtained from the radiographic imaging apparatus 20. Examples of the image processing include dynamic range compression, contrast conversion, look up table (LUT) processing, frequency enhancement, scattered radiation correction, noise suppression, image trimming, image masking, image rotation, and image inversion.

In particular, the image processor 36 functions as a pixel value correction section that performs a pixel value correction process of correcting pixel values of radiation images obtained from the radiographic imaging apparatus 20 so as not to cause density saturation. The image processor 36 also functions as an image combining section that performs an image combining process of combining the radiation images after the pixel value correction process to generate one composite image. Details of the pixel value correction process and the image combining process will be described later.

### Composite Image Obtaining Process

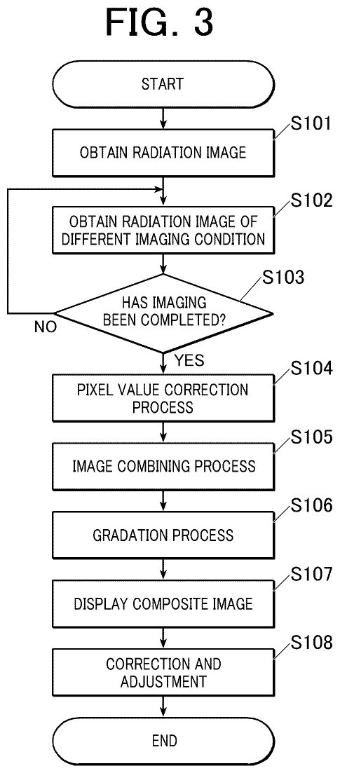

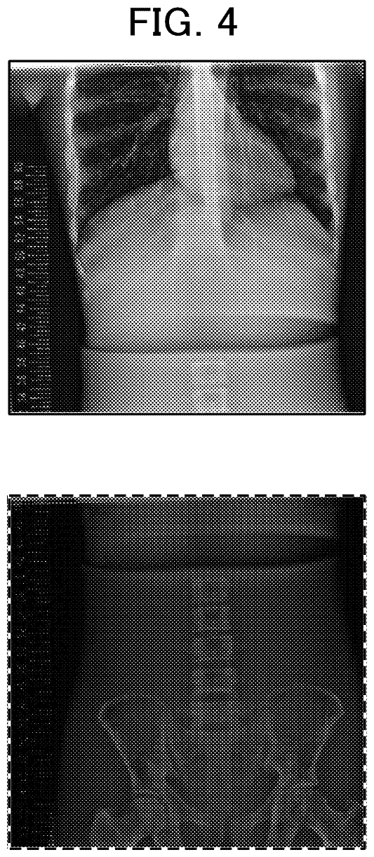

The composite image obtaining process by the radiation image processing system 100 described above will be described with reference to the flowchart of FIG. 3.

A user operates the operation part 35 of the console 30 to select imaging order information on an imaging target. The communication part 32 transmits the selected imaging order information to the radiation emission apparatus 10. The user presses the exposure switch 12 after positioning the subject. Then, radiation is emitted from the radiation source 13. The radiographic imaging apparatus 20 that has been irradiated with the radiation reads a radiation image and transmits the radiation image to the console 30 (Step S101).

After the radiation image is obtained, the user selects, with the operation part 35, imaging order information whose imaging condition is different from that of the imaging order information previously obtained (in the present embodiment, Step S101). These pieces of imaging order information differ in at least the angle of the radiation source 13 as the imaging condition. The communication part 32 transmits the selected imaging order information to the radiation emission apparatus 10. The user presses the exposure switch 12 after positioning the subject. Then, radiation is emitted from the radiation emission apparatus 10. The radiographic imaging apparatus 20 that has been irradiated with the radiation reads a radiation image and transmits the radiation image to the console 30 (Step S102).

The controller 31 that has obtained the second and subsequent radiation images displays a confirmation screen on the display part 34 as to whether radiographic imaging has been completed, and receives an instruction from the user (Step S103). If the controller 31 determines that the radiographic imaging has not been completed (Step S103; No), the process proceeds to Step S102 to obtain a radiation image on the basis of imaging order information whose imaging condition is different from that of the imaging order information more previously obtained.

If the controller 31 determines that the radiographic imaging has been completed (Step S103; Yes), the user instructs the controller 31 to perform the pixel value correction process and the image synthesis process using the operation part 35. The image processor 36 that has received the instruction to perform the pixel value correction process corrects the pixel value on the basis of the actual value of the mAs value in each piece of imaging order information (Step S104). In the pixel value correction process, the image processor 36 corrects the pixel value of each image such that the pixel value is equal to or less than the maximum value of the gradation (that is, such that density saturation does not occur).

As described above, the image processor 36 uses the actual value rather than the set value of the mAs value in this step. Therefore, for example, even in a case where an automatic exposure controller (AEC) or the like is provided and the dose changes from the imaging order information, a process according to the change can be performed. Note that the actual value of the mAs value can be obtained from the radiation emission apparatus 10.

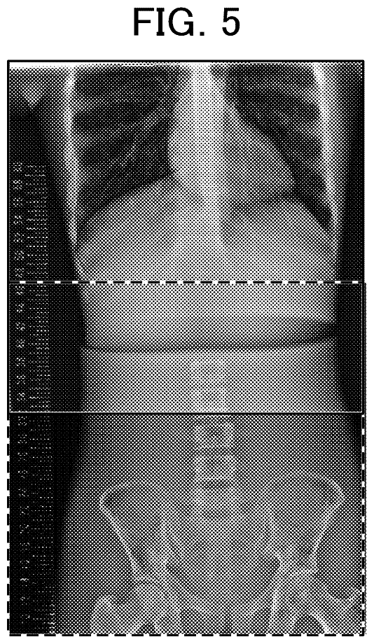

Hereinafter, as illustrated in FIG. 4, it is assumed that two radiation images, that is, a first radiation image having an mAs value of 3 and a second radiation image having an mAs value of 6, have been obtained from the radiographic imaging apparatus 20. In addition, it is assumed that the first radiation image and the second radiation image are both 16-bit images. Therefore, the maximum value of the gradation of the first radiation image and the second radiation image is 65535. Furthermore, as described above, the pixel value of the first radiation image is 1000 to 40000. Furthermore, the pixel value of the second radiation image is 2000 to 50000.

At the time, if the pixel value correction process is performed based on the mAs values such that the pixel value of the first radiation image is adjusted to the pixel value of the second radiation image, the pixel value of the first radiation image is 2000 to 80000, which may cause density saturation. Therefore, in the present embodiment, the image processor 36 performs the pixel value correction process such that the pixel value of the second radiation image is adjusted to the pixel value of the first radiation image having the lowest mAs value. Then, the pixel value of the second radiation image is 1000 to 25000, and therefore the density saturation does not occur.

Note that in a case where the pixel value becomes a decimal as a result of correcting the pixel value of the radiation image, the pixel value may be uniformly multiplied by a predetermined coefficient such that the pixel values of all the radiation images become integers. With the above-described control, occurrence of a rounding error can be suppressed, and occurrence of a density shift can be suppressed.

After the pixel value correction process, as illustrated in FIG. 5, the image processor 36 performs the image combining process of combining the radiation images to generate one composite image (Step S105). As illustrated in FIG. 4 and FIG. 5, in Step S101 and Step S102, the user captures radiation images so as to have image overlap areas. Therefore, the image processor 36 combines the radiation images such that the image overlap areas are laid on top of one another.

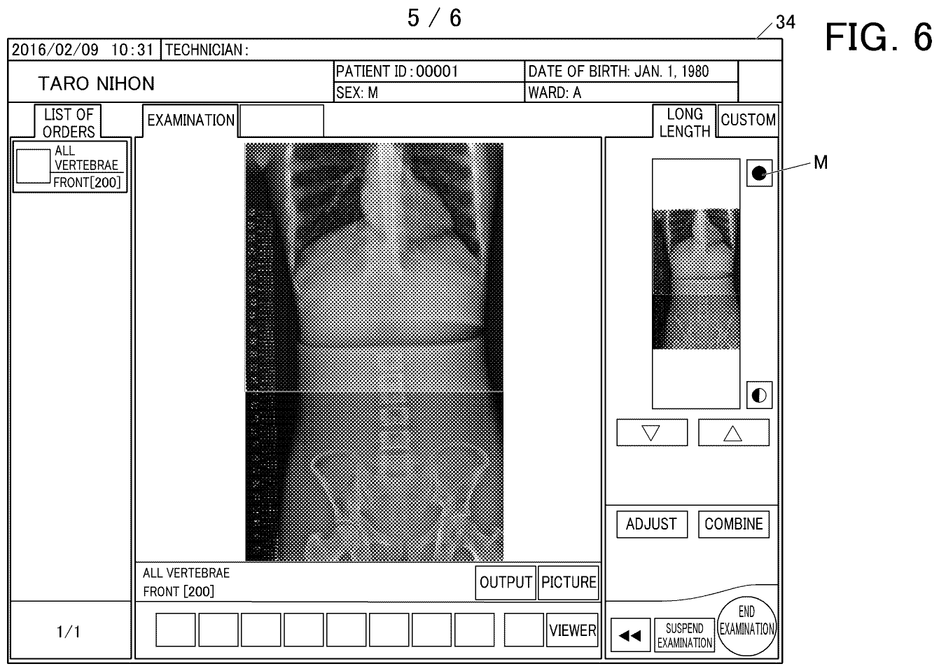

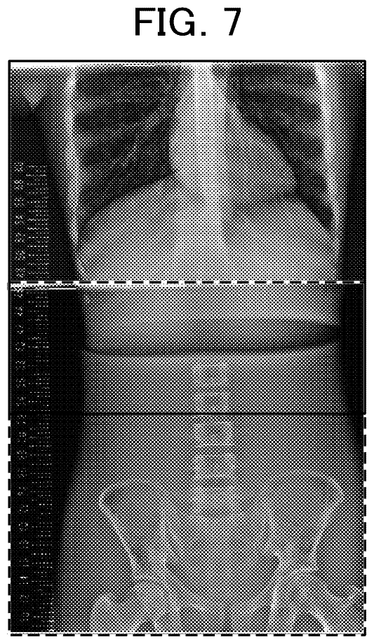

After generating the composite image, the image processor 36 performs a gradation process to adjusting the contrast of the composite image (Step S106). The controller 31 causes the display part 34 to display the composite image generated by the image processor 36 as illustrated in FIG. 6 (Step S107). The user appropriately adjusts the pixel value and the combining position regarding the displayed composite image using the operation part 35 (Step S108).

### Advantageous Effects of Embodiment

As described above, the console 30 according to the present embodiment includes the controller 31 that functions as the image obtaining section that obtains radiation images of a subject captured under different imaging conditions. The console 30 further includes the image processor 36 that functions as the pixel value correction section that corrects the pixel value of at least one radiation image among the radiation images and the image combining section that combines the radiation images to obtain one composite image. Then, the pixel value correction section corrects the pixel value of the at least one radiation image such that the maximum value of the pixel value of the at least one radiation image is equal to or less than the maximum value of the gradation of the image. Thus, with the console 30 according to the present embodiment, it is possible to obtain a composite image in which density saturation is suppressed and that can be easily interpreted while suppressing the influence of multiple times of exposure on the subject.

### Others

Although specific descriptions have been given above based on the embodiment(s) according to the present invention, the present invention is not limited to the above-described embodiment(s). Various modifications can be made within the scope of the invention described in claims and their equivalents.

For example, in the above, the image processor 36 corrects the pixel value of the second radiation image so as to match the pixel value of the first radiation image having the lowest mAs value, but not limited thereto. The method for the pixel value correction process by the image processor 36 is not specifically limited as long as the finally obtained composite image does not have density saturation. Therefore, for example, the image processor 36 may correct the pixel value of the first radiation image having a relatively low mAs value so as to match the pixel value of another radiation image having a relatively high mAs value.

Furthermore, in the above, the image processor 36 uniformly corrects the pixel value of the second radiation image so as to match the pixel value of the first radiation image, but not limited thereto. As illustrated in FIG. 7, the image processor 36 may sequentially change the adaptation degree of the image processing in other radiation images in the body axis direction.

Furthermore, in the above, Step S105 in which the image combining process is performed is performed after Step S104 in which the pixel value correction process is performed, but not limited thereto. That is, the pixel value correction process may be performed after the image combining process is performed. With the above-described configuration, the user can check the composite image first, and can more quickly determine the necessity of re-imaging due to a body motion.

Furthermore, as illustrated in FIG. 6, the controller 31 may perform emphasis display by which whether any radiation image has been used as a reference to perform the pixel value correction process in Step S107 can be recognized. In FIG. 6, an identification mark M is added as an example of the emphasis display, but the emphasis display is not limited thereto. For example, only the radiation image used as a reference may be overlaid and displayed.

Furthermore, Step S108 is not limited to manual adjustment of the composite image with the operation part 35. That is, the process may proceed to step S104 after the radiation image serving as the reference of the pixel value is selected, and the image processor 36 may be caused to perform the pixel value correction process and the image combining process again. At the time, it may be possible to adopt a configuration in which the user can select a pixel value processing method performed by the image processor 36 from the above-described types. A configuration may be adopted in which the user can select a pixel value processing method at the timing of an execution instruction of the pixel value correction process and the image combing process, namely, immediately before Step S104.

In the above, the imaging order information obtained from the RIS is stored in the storage section 33, but not limited thereto. The imaging order information may be input by a photographer operating the operation part 35.

In the above, the image processor 36 performs the pixel value correction process because radiation images have the density difference, but the present invention is not limited thereto. That is, the combining process may be performed without performing the pixel value correction process if a radiation image has substantially the same pixel value as that of a radiation image as a reference.

In the above, radiation images are obtained by changing the angle of the radiation source 13, but the present invention is not limited thereto. A configuration may be adopted in which radiation sources 13 having different irradiation angles are provided, the radiation sources 13 are caused to emit radiation based on different pieces of imaging order information, and radiation images are obtained at once. With the above configuration, it is possible to suppress a shift in radiation images due to movement of the subject between radiation emissions.

In the above, a hard disk, a nonvolatile semiconductor memory, or the like is used as a computer-readable medium storing the program(s) according to the present invention, but the computer-readable medium is not limited thereto. As the computer-readable medium, a portable recording medium such as CD-ROM can be used. Furthermore, as a medium providing data of the program according to the present invention via a communication line, a carrier wave is also applied.

Although embodiments of the present invention have been described and illustrated in detail, the disclosed embodiments are made for purposes of illustration and example only and not limitation. The scope of the present invention should be interpreted by terms of the appended claims.

What is claimed is:

1. A radiation image processing apparatus comprising:
a hardware processor that obtains radiation images of a subject captured under imaging conditions different from one another; and
an image processor that corrects a pixel value of at least one radiation image of the radiation images, and combines the radiation images to obtain one composite image,
wherein each of the radiation images has an image overlap area with one or more other radiation images of the radiation images, and
wherein the image processor corrects the pixel value of the at least one radiation image such that a maximum value of the pixel value of the at least one radiation image is equal to or smaller than a maximum value of an image gradation.

2. The radiation image processing apparatus according to claim 1,
wherein the hardware processor obtains an mAs value from each of the imaging conditions, and
wherein the image processor corrects the pixel value of the at least one radiation image based on the mAs value.

3. The radiation image processing apparatus according to claim 2, wherein based on a pixel value of a radiation image the mAs value of which is lowest among the radiation images, the image processor corrects the pixel value of the at least one radiation image.

4. The radiation image processing apparatus according to claim 1, wherein in response to the corrected pixel value of the at least one radiation image being a decimal, the image processor multiplies the pixel value of each of all the radiation images by a predetermined coefficient to make the pixel value of each of all the radiation images an integer.

5. The radiation image processing apparatus according to claim 1, wherein the hardware processor causes a display to display the composite image and the radiation images, and causes the display to display, among the radiation images, a radiation image based on which the pixel value of the at least one radiation image has been corrected, so as to be distinguishable.

6. A radiation image processing system comprising:
the radiation image processing apparatus according to claim 1;
a radiation emission apparatus that emits radiation; and
a radiographic imaging apparatus that generates a radiation image by receiving the radiation emitted by the radiation emission apparatus.

7. A radiation image processing method comprising:
obtaining radiation images of a subject captured under imaging conditions different from one another;
correcting a pixel value of at least one radiation image of the radiation images, and
combining the radiation images to obtain one composite image,
wherein each of the radiation images has an image overlap area with one or more other radiation images of the radiation images, and
wherein the correcting includes correcting the pixel value of the at least one radiation image such that a maximum value of the pixel value of the at least one radiation image is equal to or smaller than a maximum value of an image gradation.

8. A non-transitory computer-readable storage medium storing a program causing a computer to:
obtain radiation images of a subject captured under imaging conditions different from one another;
correct a pixel value of at least one radiation image of the radiation images, and
combine the radiation images to obtain one composite image,
wherein each of the radiation images has an image overlap area with one or more other radiation images of the radiation images, and
wherein the program causes the computer to correct the pixel value of the at least one radiation image such that a maximum value of the pixel value of the at least one radiation image is equal to or smaller than a maximum value of an image gradation.
